(19) United States
(12) Patent Application Publication
(10) Pub. No.: US 2013/0211207 A1
(43) Pub. Date: Aug. 15, 2013

(54) METHOD AND SYSTEM FOR MONITORING RESPIRATORY GASES DURING ANESTHESIA

(71) Applicants: Yvonne Joseph, Fellbach (DE); Isabelle Raible, Tuebingen (DE); Mustafa Sarpasan, Stuttgart (DE); Nadejda Krasteva, Fellbach (DE); Gabriele Nelles, Stuttgart (DE)

(72) Inventors: Yvonne Joseph, Fellbach (DE); Isabelle Raible, Tuebingen (DE); Mustafa Sarpasan, Stuttgart (DE); Nadejda Krasteva, Fellbach (DE); Gabriele Nelles, Stuttgart (DE)

(73) Assignee: Sony Corporation, Tokyo (JP)

(21) Appl. No.: 13/587,339

(22) Filed: Aug. 16, 2012

(30) Foreign Application Priority Data
Sep. 9, 2011 (EP) 11007357.4

(51) Int. Cl. A61B 5/08; A61M 16/12; A61B 5/097; A61M 16/10; A61B 5/01

(52) U.S. Cl. CPC A61B 5/082; A61M 16/104; A61B 5/01; A61B 5/097; A61M 16/12
USPC 600/301; 600/532

(57) ABSTRACT

An e-nose device employing chemiresistive sensors with a thin nanoparticle film allows to perform a fast and accurate analysis of respiratory gases during anesthesia in real-time over the entire breathing cycle. A combinatorial selection of nanoparticles and organic linker molecules in the nanoparticle film allows to achieve a high selectivity, which is particularly suitable for detecting a change of anesthetics or analgesics over time.

### FIELD OF THE INVENTION

The present invention relates to a method and system for monitoring respiratory gases during anesthesia by means of chemiresistive sensors, in particular with an electronic nose.

### BACKGROUND AND RELEVANT STATE OF THE ART

A state of anesthesia can be attained by employing either inhalational or intravenous anesthetics, or a combination of both. Common examples of inhalational anesthetics are halothane, enflurane, isoflurane, sevoflurane, and desflurane. Typically, N₂O as an analgesic agent is also provided to the patient together with the anesthetic agents.

Patient monitoring is important during anesthesia to ensure the patient's safety and well-being. Careful monitoring may prevent the patient from receiving either too little or too much of an anesthetic or analgesic, and may ensure a smooth transition when an anesthetic agent is exchanged, so as to monitor agent uptake, distribution and concentration.

State-of-the-art anesthetic gas monitors for inhalation anesthetics employ infrared (IR) photospectrometry. Examples of these monitors are described in US patents U.S. Pat. No. 5,932,877 and U.S. Pat. No. 5,778,874. Dispersive infrared (DIR) or non-dispersive infrared (NDIR) techniques are commonly used to measure the IR absorbance of the gases of interest. DIR employs a single optical filter and a prism or a diffraction grating to separate the component wavelengths for each agent. In contrast, NDIR employs multiple narrow-band optical filters through which the infrared emission is passed in order to determine which gas is present in the mixture. Examples of commercial IR gas monitors that have wide currency today are Datex-Ohmega (GE Healthcare, Finland), Andros/LumaSense (LumaSense, US), and Scio/Vamos (Drager Medical, Germany).

A disadvantage of IR monitors is the complex system build-up due to the usage of optical filters or gratings and IR light sources. Moreover, sophisticated mathematical data analysis is usually required to deconvolute the overlapping absorbance spectra of the inhalation anesthetic gases in the 3-15 μm wavelength range. A further disadvantage of IR monitors is the relatively high sample flow rate (typically in excess of 200 ml/min) required to achieve an acceptable response time of less than 800 ms. The high sample flow rate prohibits the use of IR analyzers with patients whose expiratory and inspiratory gas flow rates are below the analyser sampling flow rates, such as with infants. The high sample flow rate may also lead to contamination of the sample by the inspired air, or to subatmospheric pressures in the airway.

US 2010/0085067 describes an inhalational anesthetics monitor and measurement device that employs nanostructured field effect transistor sensors comprising single wall carbon nanotubes (CNT). The sensor device simultaneously measures capacitance and resistance of the CNT network in the presence of the analyte. The analyte may be identified from the capacitance/resistance ratio, and the concentration of gas in the breath may be determined from either the capacitance or resistance change. The sensitivity and selectivity of the sensors to the anesthetic agents, such as CO₂ or N₂O is achieved by introducing additional functionality to the CNT material, such as by implementing layers of selective polymers or by functionalizing the CNT surface itself. The sensors are lightweight disposable units for direct airway sampling.

Disadvantages of the sensor unit described in US '067 are the need of a complex readout circuitry as well as the slow response rate to analyte concentration changes. Demonstrated signal saturation is within the minute time frame, which will be insufficient for real-time monitoring of the anesthetic agent during a breathing cycle. Moreover, the monitor described in US '067 may measure only either the inhaled air or the exhaled air, depending on the location of the sensor in the breathing circuit, but not both.

US 2003/0176804 A1 describes a system and method for monitoring the delivery of both inhalational and intravenous anesthetic agents in the exhaled breath. The sensors may be either gravimetric, such as sensors employing surface acoustic waves (SAW), or resistive, employing conducting polymers or semiconductive oxide sensors. The analyser is an artificial olfaction (so-called “e-nose”) device that generates an electrical signal from the sensors. The presence and concentration of an anesthetic agent may be determined by using neural networks to compare the pattern of the sensor array and the change in the electrical signal (caused by either a frequency shift in the SAW sensors or by a resistance change in the resistive sensors) to a previously obtained pattern and signal magnitude for the target compound. The method described in US '804 requires the operation of two analysers, one connected to the inhaled air flow and another connected to the exhaled air flow.

In view of the shortcomings of the prior art, what is needed is a system and method for monitoring respiratory gases during anesthesia that allows a faster, and preferably real-time chemical identification of the anesthetic agent as well as a reliable quantitative determination of its concentration in both inhaled and exhaled breath samples.

### OVERVIEW OF THE PRESENT INVENTION

This objective is achieved with a system and method for monitoring respiratory gases according to independent claims 1 and 11, respectively. The dependent claims relate to preferred embodiments.

A system for monitoring respiratory gases according to the present invention comprises sensor means comprising a sensor array with a plurality of chemiresistive sensors, as well as conduct means adapted for supplying, to said chemiresistive sensors, an inhaled gas of a subject and/or an exhaled gas of said subject. Said sensor means are adapted to monitor said inhaled gas of said subject and/or said exhaled gas of said subject by analysing a gas sample supplied via said conduct means to said chemiresistive sensors, in particular adapted to monitor a composition and/or concentration of said inhaled gas and/or a composition and/or a concentration of said exhaled gas. Said chemiresistive sensors comprise a nanoparticle film with a nanoparticle network, said nanoparticle network formed of nanoparticles interlinked through linker molecules, in particular organic linker molecules, wherein a thickness of said nanoparticle film is no greater than 100 nm.

In a preferred embodiment, said thickness of said nanoparticle film is no greater than 75 nm, particularly no greater than 50 nm. Preferably, said nanoparticle film has a thickness no greater than 25 nm.

In particular, each said chemiresistive sensor may comprise a nanoparticle film as described above.

It is the realisation of the inventors that a nanoparticle film with a nanoparticle network formed of nanoparticles interlinked through linker molecules at the specified thickness range allows to provide a system for monitoring respiratory gases that combines a high selectivity with a fast response time. In particular, a thickness of said nanoparticle film that does not exceed 100 nm allows the analytes to permeate said nanoparticle film easily, so that the anesthetic agent can be identified quickly, and its concentration can be quantitatively determined quickly. In particular, the system according to the present invention allows to achieve response times of well below 800 ms.

Due to the fast response times, the system according to the present invention allows to perform a real-time analysis of the respiratory gases over the whole breathing cycle, including both inhalation and exhalation.

Since the analyte to be detected may permeate the nanoparticle film quickly, a small sample flow is sufficient to detect and identify anesthetics and/or analgesics with a high decree of accuracy. This is a significant advantage when monitoring patients with a compararatively low inhalation and exhalation gas flow, such as infants or children.

In general, said subject may be a human or animal patient, for instance a patient undergoing surgery.

In a preferred embodiment, said nanoparticle network of at least one of said chemiresistive sensors, in particular said nanoparticle networks in all of said chemiresistive sensors, does not comprise a polymer. The inventors found that comparatively small linker molecules are particularly suited to enable a quick penetration of the analytes into the nanoparticle film, thereby supporting fast response times.

However, non-linear polymer molecules or oligomer molecules, in particular dendrimer molecules were also found suitable, and may be comprised in at least one of said chemiresistive sensors, preferably in all of said plurality of chemiresistive sensors.

Preferably, said linker molecules may be bi- or poli-functional organic molecules.

Interlinking functional groups may preferably be —SH (thiol)′; —N—CSS (dithiocarbamate); —NH2 (amine); —COOH (carboxy); —SCN (thiocyanate); and/or —C—S—S—C— (disulphide).

Preferably, the terminal linking group may be chosen so to allow the formation of a strong covalent or electrostatic bond between the nanoparticle and the linker molecule.

In a preferred embodiment, S-containing functional groups are selected as terminal linking groups for Au or Ag, whereas N-containing functional groups may be selected as terminal linking groups for Pt, and O-containing groups may be selected as terminal linking groups for Fe or Cu.

In a preferred embodiment, said nanoparticles in said nanoparticle network are arranged in a plurality of layers, wherein nanoparticles of adjacent layers are connected by said linker molecules.

By forming said nanoparticle film layer-by-layer, for instance in a self-assembly process, a very thin and homogeneous film with excellent penetration properties can be obtained.

In a preferred embodiment, said nanoparticles are metallic nanoparticles, and in particular comprise a metal selected from the group consisting of Au, Pt, Ag, Pd, Cu, Ni, Cr, Mo, Zr, Nb, Fe, or any combination of these metals.

Nanoparticle films according to the latter embodiments are generally known in the art of sensor arrays, and have been described in the related patent applications EP 1 022 560 A1, EP 1 278 061 A1, and EP 1 215 485 A1. However, these previous applications were not in any way related to the monitoring of respiratory gases during anesthesia. In fact, it is the realisation of the inventors that thin nanoparticle films employing a nanoparticle network of nanoparticles interlinked through linker organic molecules are particularly suited for monitoring anesthetics and/or analgesics in an inhaled gas and/or an exhaled gas of a patient, and allow to achieve a quick and reliable determination of the concentration of these gases as well as a high level of selectivity.

In a preferred embodiment, said sensor array comprises a first group of chemiresistive sensors comprising hydrophilic linker molecules, and/or a second group of chemiresistive sensors comprising hydrophobic linker molecules, and/or a third group of chemiresistive sensors comprising amphiphilic linker molecules.

The inventors found that by providing different groups of chemiresistive sensors comprising materials with different solubility (hydrophilicity) properties the selectivity of the sensor means can be greatly enhanced.

For hydrophobic linker molecules, preferably only C-containing chains may be employed, in particular alkane, alkene, and/or aromatics.

In a preferred embodiment, the number of C-atoms in the linker molecule is no smaller than 5, preferably no smaller than 10.

In a further preferred embodiment, the number of C-atoms in the linker molecules in no larger is than 20.

For hydrophilic linker molecules, functional groups along the C-chain are preferably implemented. These may comprise amide, alcohol, carboxyl, amine, ether, and/or ester groups.

Preferably, the length of pure C-C segments in the chain of the linker molecules is no larger than 6, in particular no larger than 3 C-containing groups in a row.

In a preferred embodiment, F-functionalities and/or Cl-functionalities may be provided in the linker molecules along the alkyl chain.

This allows to increase the selectivity of the sensors to inhalation anesthetics, which are often fluorinated compounds. These functionalities were found to result in an increased hydrophobicity as well as an increased donor-acceptor interaction with the anesthetics.

Preferably, the sensor material of said chemiresistive sensors may be selected such that the interaction between the anesthetics and/or analgesics and the sensor material is reversible. In particular, the sensor material may be selected to return to an initial resistance level after removal of the anesthetics analyte.

In the latter embodiment, no loss of anesthetics or analgesics, and thus no concentration change is caused by the measurement. This is a significant advantage over sensors operating via burning or destroying the analyte, as well as over sensors responding irreversibly to the analyte, such as semiconductive metal oxide chemiresistors or electrochemical sensors.

A reversible sensor has the further advantage that no poisoning of the sensor may occur, and that no toxic or health-adverse products of the anesthetics and/or analgesics are formed and transported further with the gas flow.

Preferably, said conduct means are adapted for connecting said sensor array to an inlet conduct, said inlet conduct for supplying an anesthetic gas and/or an analgesic gas to said subject, and/or connecting said sensor array to an outlet conduct, said outlet conduct for removing exhaled breath from said subject.

In particular, said conduct means may be adapted for sidestream connection to an inlet conduct, said inlet conduct for supplying an anesthetic gas and/or an analgesic gas to said subject, and/or for sidestream connection to an outlet conduct, said outlet conduct for removing exhaled breath from said subject.

As described above, in the present invention very small sample sizes suffice to provide a reliable and quick analysis of the respiratory gases. Hence, only small quantities of the inhaled gas and/or the exhaled gas need to be supplied to the sensor array, which makes the invention ideally suited for use in a sidestream connection.

A sidestream connection has the advantage of minimal interference with the breathing circuit to which said inlet conduct and said outlet conduct may be connected. This allows to provide an integrated system in which said sensor means and conduct means are combined with analysation means for performing the analysis of said inhaled gas and/or said exhaled gas, and which can be connected to any conventional breathing circuit by means of said sidestream connection.

Sidestream connections are sometimes associated with a slow response time. However, the particular composition of the chemiresistive sensors according to the present invention allows to analyse the inhaled gas and/or exhaled gas quickly, thereby outweighing any delay that may be experienced as a result of the sidestream connection.

In a preferred embodiment, said sidestream connection may be established by means of a conduct means comprising a branch line in fluid communication with said sensor array, said branch line adapted for connecting said sensor array to said inlet conduct and/or to said outlet conduct.

However, the system according to the present invention is by no means limited to a sidestream connection. In an alternative embodiment, said conduct means may be adapted for mainstream connection to an inlet conduct, said inlet conduct for supplying an anesthetic gas and/or an analgesic gas to said subject, and/or for mainstream connection to an outlet conduct, said outlet conduct for removing exhaled breath from said subject.

In particular, said conduct means may be adapted for connecting said sensor array such that said sensor array forms part of a breathing circuit of said subject or may be integrated in a breathing circuit of said subject, such that an inhaled gas supplied to said subject passes said chemiresistive sensors and an exhaled gas removed from said subject passes said chemiresistive sensors.

A mainstream connection in which the sensor array is placed directly in the breathing circuit has the advantage that it allows to perform a particularly fast analysis of the inhaled gas and/or exhaled gas.

In a preferred embodiment, said sensor means comprise a humidity sensor and/or a temperature sensor, and/or CO₂ and/or O₂ sensors.

Said humidity sensor and/or said temperature sensor may be provided within or integrated into said sensor array, so that said humidity sensor and/or said temperature sensor are surrounded by said chemiresistive sensors. This allows to adjust the analysis of the inhaled gas and/or exhaled gas by taking into account the humidity and/or temperature of the respiratory gas, with high accuracy.

In a preferred embodiment, said temperature sensor may be a sealed sensor, i.e., a sensor sealed against the gas stream.

Said temperature sensor may otherwise be identical to the chemiresistive sensors used for gas detection, and in particular may be made from the same material and/or according to the same fabrication technique.

According to a preferred embodiment, said conduct means may further comprise a reference inlet in fluid communication with said sensor array, said reference inlet adapted for supplying a reference gas sample to said chemiresistive sensors.

Said reference gas sample may be an ambient air sample or may be a gas sample of a predefined composition, in particular a gas with a predefined concentration of an anesthetic and/or analgesic.

It is the realisation of the inventors that a reference gas sample advantageously allows to perform a calibration of the sensor means, either initially before starting to monitor the respiratory gases, or at predetermined time intervals during the monitoring, or continuously during the monitoring. This allows to adjust the sensor device to varying ambient conditions, such as humidity conditions, and/or to reduce the cross-sensitivity to other components of the respiratory gases. This ensures a high level of accuracy and reliability even if the sensor device is moved together with the patient, for instance from an operating theatre to a waking room.

In a preferred embodiment, said conduct means may comprise an integrated exchanger tube adapted for letting said inhaled gas and/or said exhaled gas exchange humidity with said reference gas sample. This provides a simple, and yet very effective means to adjust the humidity of the breath sample to the humidity of the reference gas.

In a preferred embodiment, said conduct means may comprise a valve for selectively connecting either said inlet conduct or said outlet conduct or said reference inlet to said sensor array.

The conduct means may preferably comprise a filter device adapted for filtering said inhaled gas and/or said exhaled gas and/or said reference gas sample. This allows to minimize the influence of volatile organic components in the breath of the subject.

According to a preferred embodiment, said conduct means comprise a flow means for setting a gas flow through said conduct means and/or said reference inlet to a predetermined flow rate.

In particular, said conduct means may comprise an orifice with a variable flow diameter.

In a preferred embodiment, said sensor means comprise analysation means adapted to identify an anesthetic and/or analgesic in said inhaled gas and/or in said exhaled gas, and/or adapted to determine a concentration of an anesthetic and/or an analgesic in said inhaled gas and/or said exhaled gas by determining a ratio of a signal provided by a first chemiresistive sensor to a signal provided by a second chemiresistive sensor, said second chemiresistive sensor different from said first chemiresistive sensor.

It is the realisation of the inventors that a reliable analysis of the inhaled gas and/or exhaled gas may be achieved by comparing the signals provided by different chemiresistive sensors. This is a simple, yet effective technique, which can be performed very quickly and which contributes to the enhanced sample rate achieved in the system according to the present invention.

The invention likewise relates to a method for monitoring respiratory gases during anesthesia, comprising the steps of providing sensor means comprising a sensor array with a plurality of chemiresistive sensors, exposing said chemiresistive sensors to an inhaled gas of a subject and/or to an exhaled gas of said subject, and analysing said inhaled gas and/or said exhaled gas. Said chemiresistive sensors comprise a nanoparticle film with a nanoparticle network, said nanoparticle network formed of nanoparticles interlinked through linker molecules, wherein a thickness of said nanoparticle film is no greater than 100 nm, preferably no greater than 50 nm.

In particular, said method may comprise the steps of monitoring, in said inhaled gas and/or said exhaled gas, a concentration of an anesthetic and/or analgesic, and/or detecting, in said inhaled gas and/or said exhaled gas, volatile gases different from an anesthetic or analgesic, and/or detecting in said exhaled gas, a metabolic product of an anesthetic and/or analgesic.

It is a significant advantage that the present invention allows for the monitoring of anesthetics and/or analgesics in both inhaled and exhaled breath in a single sensor array.

In a preferred embodiment, said chemiresistive sensors are alternately exposed to an inhaled gas sample of said subject and to an exhaled gas sample of said subject.

This allows to monitor the respiratory gases of said subject over the entire breathing cycle.

In a preferred embodiment, the method further comprises the steps of exposing said chemiresistive sensors to a reference gas sample, and comparing said inhaled gas and/or said exhaled gas to said reference gas sample.

Preferably, said chemiresistive sensors may be consecutively exposed to said inhaled gas of said subject and to said exhaled gas of said subject and to said reference gas sample, in any desired order.

According to a preferred embodiment, said chemiresistive sensors may be exposed to said reference gas sample after a predetermined number n of breathing cycles, each said breathing cycle comprising the steps of consecutively exposing said chemiresistive sensors to said inhaled gas of said subject and to said exhaled gas of said subject.

In a preferred embodiment, n≧5, particularly n≧10. This minimizes the dead time associated with the analysis of the reference gas, while still allowing to provide a reliable reference.

In an alternative embodiment, said chemiresistive sensors are exposed to said reference gas sample after each breathing cycle. This allows to perform a particularly accurate identification of the anesthetic and/or analgesic in the respiratory gases, as well as their respective concentrations.

According to a preferred embodiment, the method further comprises the step of establishing a contact of said inhaled gas and/or said exhaled gas with said reference gas sample, preferably via an integrated exchanger tube, so to allow said inhaled gas and/or said exhaled gas exchange humidity with said reference gas sample.

Preferably, said method may comprise a step of measuring a humidity of said inhaled gas and/or said exhaled gas and/or said reference gas sample.

In a further preferred embodiment, the method comprises a step of measuring a temperature of said inhaled gas and/or said exhaled gas and/or said reference gas sample.

The method may further comprise a step of setting a gas flow of said inhaled gas and/or said exhaled gas and/or said reference gas sample to a predetermined flow rate.

In a preferred embodiment, the method comprises a step of identifying an anesthetic and/or analgesic in said inhaled gas and/or said exhaled gas, and/or the step of determining a concentration of an anesthetic and/or analgesic in said inhaled gas and/or said exhaled gas by determining a ratio of a signal provided by a first chemiresistive sensor to a signal provided by a second chemiresistive sensor, said second chemiresistive sensor being different from said first chemiresistive sensor.

The method according to the invention may preferably employ a system with some or all of the features described above.

The invention further relates to a storage means adapted for storing computer-readable instructions, such that said instructions, when read on a computer connected to a system for monitoring respiratory gases with some or all of the features as described above, implement on said system a method with some or all of the features as described above.

### DETAILED DESCRIPTION OF PREFERRED EMBODIMENTS

The features and numerous advantages of the present invention will best be understood from a detailed description of the preferred embodiments with reference to the accompanying figures, in which:

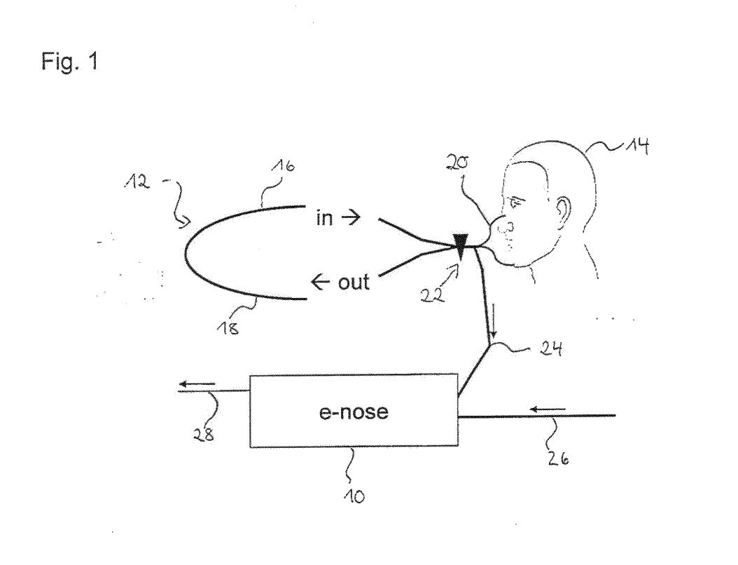

FIG. 1 schematically illustrates a system for monitoring respiratory gases according to an embodiment of the present invention, when connected to a breathing circuit of a patient under surgery;

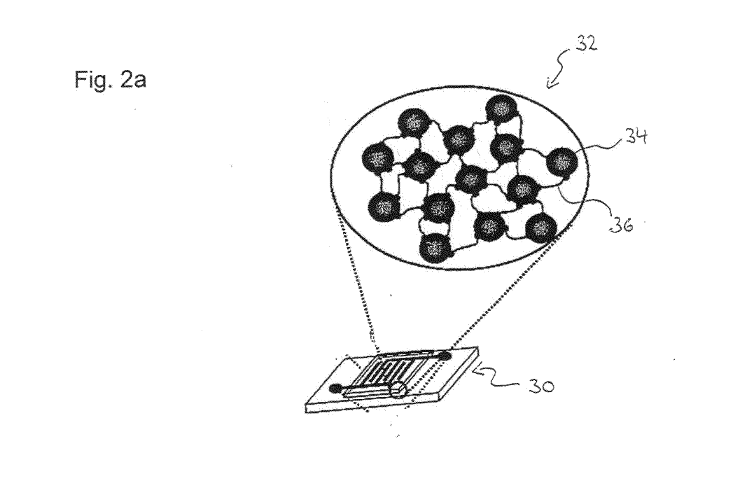

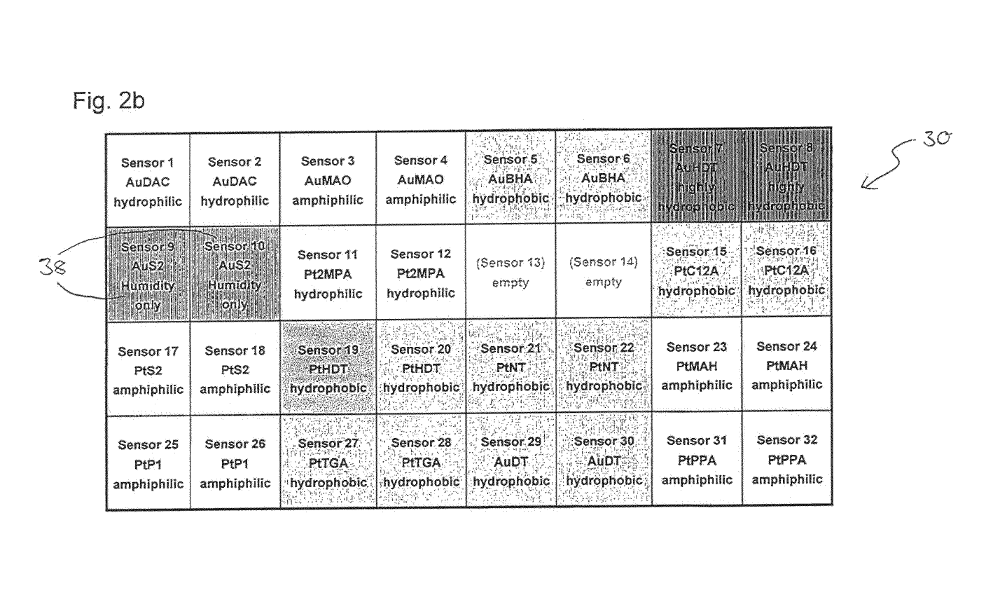

FIG. 2a is a schematic representation of a chemiresistive sensor according to an embodiment of the present invention;

FIG. 2b is an exemplary materials map of a sensor array according to an embodiment of the present invention;

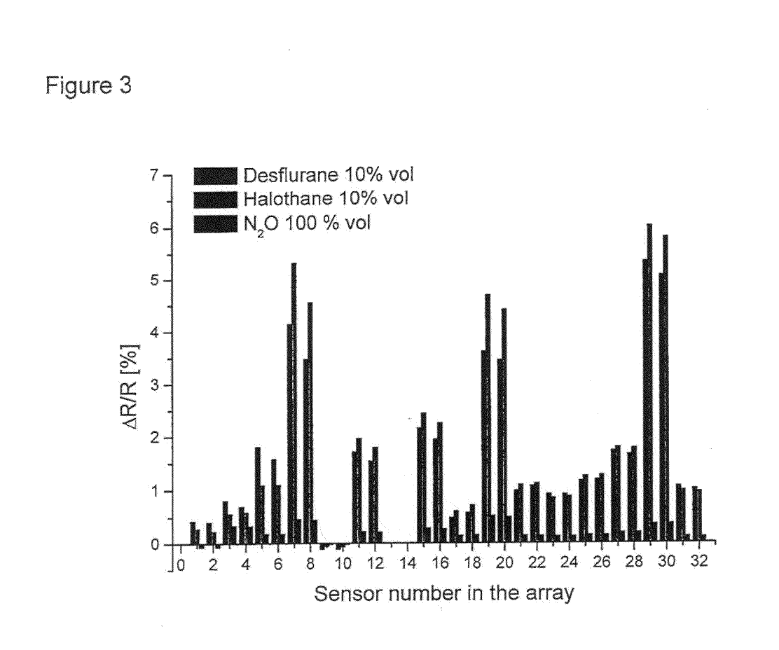

FIG. 3 is a diagram showing the relative change of resistance in the different sensors of a sensor array according to the present invention, for three different inhalation anesthetics in comparison;

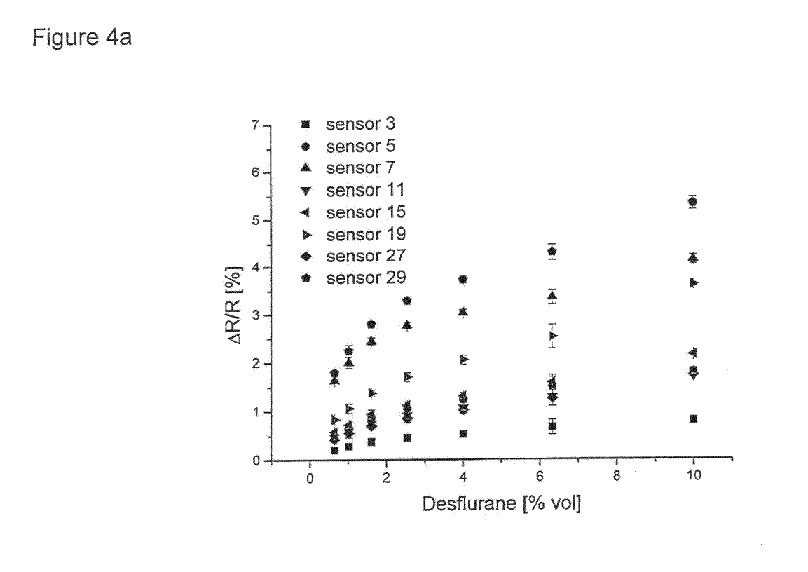

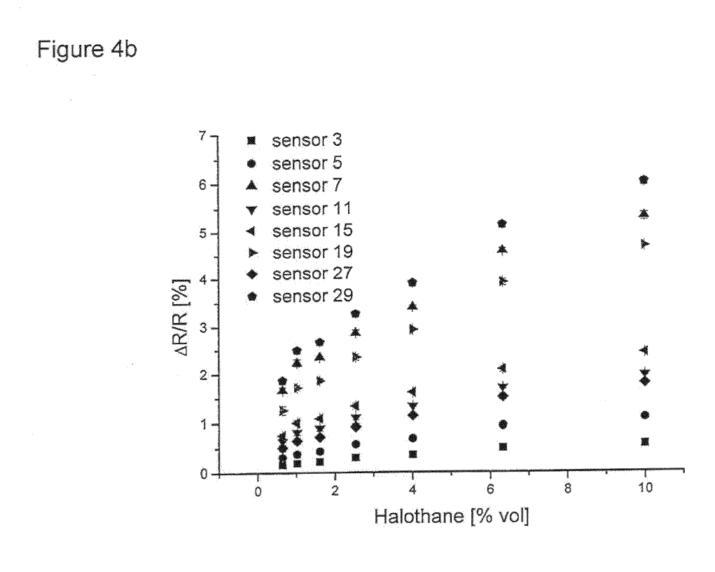

FIG. 4a illustrates the relative change of resistance for different sensors in a sensor array according to an embodiment of the invention, as a function of on the concentration of desflurane;

FIG. 4b shows a diagram corresponding to the diagram of FIG. 4a, but for different concentrations of halothane;

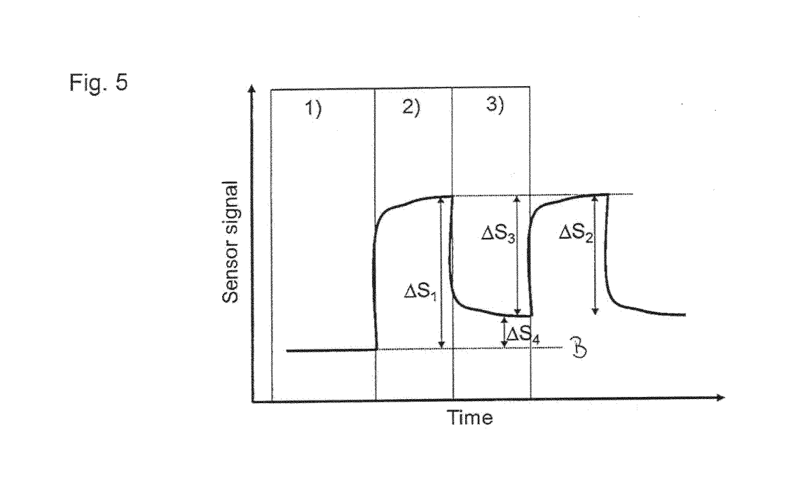

FIG. 5 schematically illustrates a sensor signal proportional to the anesthetic concentration over time, in a breathing cycle of a patient under surgery;

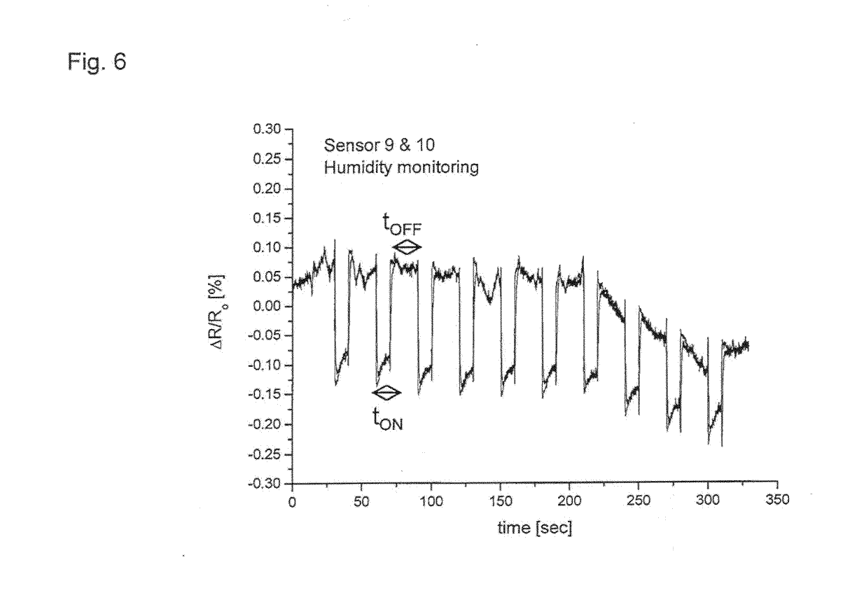

FIG. 6 graphically illustrates the relative change of resistance detected by the humidity sensors in a sensor array according to an embodiment of the invention, when alternately exposed to a desflurane sample and an ambient air reference sample; and

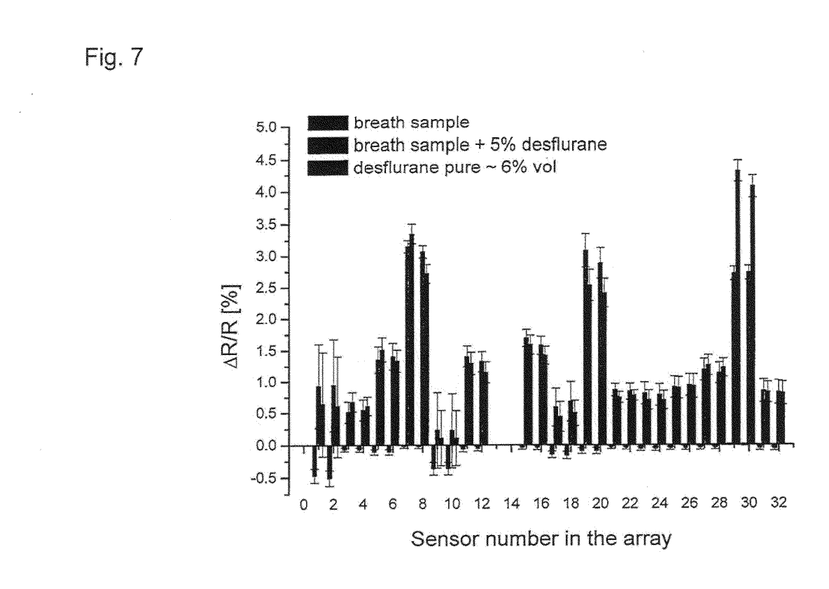

FIG. 7 illustrates the cross-sensitivity to breath matrix components in an array pattern comparison of a breath sample with desflurane and pure desflurane, for the different sensors in a sensor array according to an embodiment of the present invention.

An embodiment of the present invention relates to a system for monitoring respiratory gases during anesthesia. Electronic devices intended for the detection and/or analysis of gases are sometimes referred to in the art as “electronic noses” or “e-noses”, and this terminology will also be employed in the description of the preferred embodiments.

FIG. 1 shows an e-nose device 10 connected to a breathing circuit 12 of a patient 14 undergoing surgery. The breathing circuit 12 comprises an inlet line 16 for supplying an inhalation gas to said patient. The inhalation gas may comprise any inhalational anesthetics and/or analgesics, such as halothane, enflurane, isoflurane, sevoflurane, and desflurane. The breathing circuit 12 further comprises an outlet line 18 over which exhaled gases are removed from the patient 14 and returned to a gas source (not shown). The inlet line 16 and outlet line 18 are connected to a mouth piece and/or a nose piece 20 by means of a respiratory valve 22.

FIG. 1 shows the e-nose device 10 in a sidestream configuration, in which the e-nose device 10 is connected to the breathing circuit 12 via a branch line 24 branching off from the respiratory valve 22. However, the invention is not limited to sidestream sampling, and may alternatively comprise an e-nose device 10 arranged in a mainstream configuration (not shown), so that the inlet line 16 and outlet line 18 of breathing circuit 12 both pass directly through the e-nose device 10.

Returning to FIG. 1, the branch line 24 serves to provide samples of an inhaled gas of the patient 14 from the breathing circuit 12 to the e-nose device 10 so that they may be analysed and monitored.

The e-nose device further comprises a reference inlet 26 for providing a reference gas sample from an external supply (not shown) to the e-nose device 10, so that the inhaled gas of the patient 14 and the exhaled gas of the patient 14 may be compared against the reference gas. The reference gas may be an ambient air or a gas supply of a predetermined composition, for instance a gas supply comprising predefined anesthetics and/or analgesics at preset concentration levels.

An exhaust line 28 serves to remove the gas samples from the e-nose device 10 after analysis.

The e-nose device 10 as shown in FIG. 1 comprises an array of chemiresistive sensors, such as the array 30 schematically shown in FIG. 2a. The sensor array 30 comprises a plurality of chemiresistive sensors, wherein each sensor comprises a nanoparticle film with a nanoparticle network 32 schematically shown in the inserted blowup of FIG. 2a. The nanoparticle network 32 is formed of nanoparticles, such as Au nanoparticles 34 which are arranged in layers and are interlinked by means of organic linker molecules 36.

Chemiresistive sensors of this type are described in further detail in the related applications EP 1 022 560 A1, EP 1 215 485 A1, and EP 1 278 061 A1, and reference is made to these documents for further details regarding their composition and manufacturing.

The nanoparticle network 32 forms a thin film with a thickness that is preferably no greater than 50 nm, and may be as low as a few nanometers. Such thin films can be achieved by means of a layer-by-layer self-assembly. Spotting or spraying techniques may likewise be employed to tune the structure and signal response of the sensing layer.

Analytes present in the inhaled gas sample and/or exhaled gas sample are supplied to the sensor array 30 via the branch line 24, and penetrate through the nanoparticle film to couple to the organic linker molecules 36. This changes the electric resistivity of the nanoparticle film in a way that is characteristic of the analyte. Measuring the relative change of resistance ΔR/R thereby allows to identify the analyte and to determine its concentration. The sensor selectivity may be enhanced by providing a combinatorial selection of metallic nanoparticles 34 and organic linker molecules 36, such as hydrophilic, hydrophobic, and amphiphilic sensors. The enhanced selectivity is particularly advantageous when different kinds of anesthetics and/or analgesics are consecutively supplied to a patient 14, which may be the case during different phases of a surgery.

An exemplary configuration of a sensor array 30 comprising 32 chemiresistive sensors arranged in a matrix is shown in FIG. 2b. The following abbreviations are used for the organic linker molecules in FIG. 2b:

| | |
|---|---|
| DAC—1,4,10,13-Tetraoxa-7,16- | NT—nonane dithiol |
| bisdithocarbamate-cyclooctadecan | MAH—1,6- |
| MAO—1,8-bisamidomethylthiol-octane | bisamidomethylthiol-hexane |
| BHA—N,N′-Dibutyl-1,6- | P1—Poly(propylene imine) |
| hexanebisdithiocarbamate | dendrimer generation 1 |
| HDT—hexadecane dithiol | TGA—1,11-Diamino-3,6,9- |
| S2—poly (amidoamine) dendrimer | trioxaundecane |
| generation 2 | DT—dodecane dithiol |
| 2MPA—1,5-Diamino-2-methylpentane | PPA—1,4-Bis(3- |
| C12A—dodecane diamine | aminopropyl)piperazine |

By making a suitable combinatorial selection of the nanoparticles 34 and organic linker molecules 36 and by providing these nanoparticles 34 and organic linker molecules 36 in a thin nanoparticle network 32 of a thickness of 100 nm or below, an e-nose device 10 with a high selectivity and a fast response rate can be achieved. As a general rule, the thinner the nanoparticle film, the quicker may analytes penetrate through the nanoparticle network and trigger a perceptible change of the resistivity of the chemiresistive sensor. The sensor arrays according to the present invention allow to achieve a fast chemical identification of the anesthetic and/or analgetic agents, and in particular allow to perform a real-time quantitative determination of the amount or concentration of an anesthetic or analgesic in both exhaled and inhaled breaths in the concentration range between 0 and 10% vol. The e-nose device 10 may also comprise a temperature sensor (not shown) for determining a temperature of the inhaled and/or exhaled air sample. Short response times in the range of t₉₀<800 ms, and even t₉₀<500 ms, may be achieved.

In a sensor array 30 such as the one shown in FIGS. 2a and 2b, some chemiresistive sensors may be provided redundantly. This provides a robust e-nose device 10 that may still be used to monitor respiratory gases reliably even if some chemiresistive sensors should fail, and at the same time allows to increase the signal/noise ratio. Mathematical algorithms like differentiation, multiplication or subtraction of the signals of the same type of sensors may be employed to enhance the reliability of the sensor signals.

Chemiresistive sensors typically have a resistance in the range of 10 kOhms to 2.5 MOhms.

The chemiresistive sensors of the sensor array 30 may be deposited on plastic substrates. This allows to provide non-expensive sensor arrays 30 which may be disposable, or may be personalized (patient-designated).

As can be seen from FIG. 2b, the sensor array 30 likewise comprises two humidity sensors 38 integrated into the sensor array 30 and formed from the same material. The humidity sensors 38 allow to monitor a possible dehydration of the patient 14 during anesthesia treatment.

Gas fluidics components inside the e-nose device 10 and for connection to the breathing circuit 12 may be made from chemically inert Teflon, which is particularly suitable for medical applications. Short gas paths as well as small dead volumes of fluidic compartments likewise assist to achieve a fast analysis.

The e-nose device 10 may further comprise an integrated pump as well as a valve to switch between a test sample provided from the patient 14 via the branch line 24, and a reference gas sample provided via the reference inlet 26.

The e-nose device 10 also allows to accommodate a variable gas sample flow, which may be adjusted between 10 ml/min and 300 ml/min, for instance by using an orifice with a variable diameter opening, such as an iris valve, or a pump with variable pumping volume. The sensor configuration according to the preferred embodiment allows to reliably identify and monitor anesthetics and/or analgesics in said inhaled gas sample and/or exhaled gas sample from very small sample sizes. This is a significant advantage over conventional IR monitors and conventional chemiresistive sensors alike.

Auxiliary filters, such as for the filtering of gaseous components from the sample gas or bacteria filters, may likewise be employed. The device may also comprise an integrated exchanger tube, such as a Nafion tube of a length of 20 cm, for allowing an equilibration of humidity between the breath sample and the reference gas.

Other components for preconcentration and separation of breath mixture components for sample gas pre-treatment may likewise be employed.

The e-nose device 10 further comprises electronics and processors for sensor signal acquisition and data treatment. All the data analysis software may likewise be implemented on the e-nose device 10, which thereby integrates all the components necessary for providing a real-time analysis of the amount/concentration of the respiratory gases in both exhaled and inhaled breath. The electronics and processors for sensor signal acquisition and data treatment and the data analysis software integrated into the e-nose device 10 help to achieve a fast data processing.

However, in an alternative configuration the analyis may be performed on an external computer device connected to the e-nose device 10 by means of a cable connection or wireless connection.

The array of 32 chemical sensors and the temperature sensor may be read out sequentially, employing high precision resistance measurements with an accuracy of +/−10 Ohms in the 100 kOhms to 2.5 MOhms range.

The electronics for data analysis may comprise a 6-layer printed circuit board, two microcontrollers (one for data acquisition, one for data analysis) as well as means for communication with an external memory or analysation means, such as via a USC cable connection or a wireless Ethernet connection.

All these components may be combined in a lightweight (smaller than 1.5 kg) and portable device of relatively small dimensions (roughly 300×180×50 mm, in a preferred embodiment). This allows to place the sensor in the vicinity of the patient 14 during anesthesia, and also allows to move the device together with the patient to be waking room, such as for after-anesthesia monitoring of follow-up medication monitoring.

An exemplary technique of identifying anesthetics and/or analgesics in an inhaled or exhaled gas sample, and of determining the respective concentrations will now be described with reference to FIGS. 3 and 4.

The chemical identification of an anesthetic and/or analgesic agent at the beginning of the treatment cycle can be achieved by comparing the pattern of resistance signals provided by the chemiresistive sensors of sensor array 30 to a predetermined pattern derived from preceding calibration measurements with the target anesthetic/analgesic mixtures. Exemplary sensor array patterns for desflurane and halophane as well as N₂O obtained with the sensor configuration of FIG. 2b are shown in FIG. 3. As can be taken from FIG. 3, different sensors respond differently to different types of agents.

Recognition and classification algorithms such as KDA (Kernel Discriminant Analysis) or PCA (Principal Component Analysis) can be used, followed by neural networks (NN) or artificial neural networks (ANN) analysis.

However, the inventors found that a reliable chemical identification of the anesthetic and/or analgesic agents in the course of the treatment can likewise be achieved by comparing the ratio of the signal responses of a selected subset of sensors. For instance, as can be seen from the diagram of FIG. 3, the ratio of the relative change of resistance ΔR/R is manifestly different for desflurane and halothane for sensors 3/4, 5/6, 7/8, 19/20, or 29/30. Hence, a comparison of the ratio of the relative change of resistance ΔR/R for selected subsets of sensors allows for a quick real-time determination of the type of anesthetics without having to use sophisticated mathematical algorithms. This likewise helps to speed up the analysis of the sample gases.

A quantitative determination of the anesthetic agent and/or analgesic agent concentration can be performed by comparing the magnitude of the responses of the relative change of resistance ΔR/R of selected sensors in the array to calculated data. FIGS. 4a and 4b show examples for response isotherms for desflurane (FIG. 4a) and halophane (FIG. 4b) for different concentrations, ranging from 0.5% vol to 10% vol. The data in FIGS. 4a and 4b has been obtained with the sensor array 30 shown in FIG. 2b, employing ambient air as a reference gas. As can be taken from FIG. 4a and FIG. 4b, the relative change of resistance ΔR/R generally increases with the concentration of the anesthetic agent, and careful calibration allows to determine the concentration from the resistance signals with high accuracy.

The system and method according to the present invention allows to perform the measurement and analysation of the respiratory gases continuously, over the entire breathing cycle of the patient 14. The variation of the magnitude of the sensor signals provided by the chemiresistive sensors in the inhalation phase and exhalation phase (with reference to an initial baseline signal) may be used for estimating the amount of anesthetic delivered to the patient and ingested by the patient.

FIG. 5 shows a schematic diagram of the sensor signal over time as can be recorded with an e-nose device 10 according to the present invention.

In an initial phase before start of anesthetic administration, the sensor signal (proportional to the concentration of anesthetics in the sample) is at a base level B (phase 1). The base level defines a reference to an ambient air or pre-defined test gas.

In an inhalation step (phase 2), the sensor signal rises to a level ΔS₁ above the base line B, in accordance with the rising concentration of anesthetics in the inhalation sample.

In an exhalation step (phase 3), the sensor signal falls by an amount ΔS₃, which is proportional to the difference in concentration of anesthetics between inhaled and exhaled air. The signal difference ΔS₄=ΔS₁−ΔS₃ is proportional to the difference in concentration of anesthetics between exhaled air and initially inhaled air (baseline B), and thus represents the amount of anesthetics ingested by the patient in the first breathing cycle.

The cycle then begins anew with a further inhalation step. After some time, a constant signal drop ΔS₂ corresponding to the difference in concentration of anesthetics between initial inhaled air and exhaled air will be seen in each exhalation phase.

The e-nose device 10 according to the preferred embodiment likewise allows for a straightforward humidity adjustment. Typically, the breath sample has a humidity of almost 100%. The adjustment of the breath sample humidity to the humidity of the reference gas sample (such as ambient air or a predefined reference gas) can be performed by using an integrated exchanger tube (not shown) on the breath sample path. A Nafion tube of a length of ca. 20 cm may be used for this purpose. The equilibration of the humidity can be monitored in real-time employing the humidity sensors 38 in the sensor array 30 shown in FIG. 2b.

An exemplary diagram showing the equilibration of humidity during a measurement of desflurane in 100% humidified synthetic air to the ambient air humidity is shown in FIG. 6. In FIG. 6, t_{ON} denotes the time of exposing the sensor array 30 to the desflurane sample, whereas t_{OFF} is the time of exposing the sensor array 30 to the ambient air reference with 38% humidity. Ten repetitive exposures were performed to check reproducibility.

The influence of other components in the breath matrix can likewise be minimized by using the humidity exchanger, but can also be achieved by means of specially designed filters (not shown) in the gas fluidics of e-nose device 10. These filters may filter out volatile organic components in the breath of the patient 14. Exemplary results are illustrated in FIG. 7, which shows the relative change ΔR/R of the resistance of sensors 1 to 32 for a breath sample without desflurane, a breath sample with 5% desflurane, and pure desflurane at about 6% vol. in comparison. As can be taken from FIG. 7, the signals of sensor array 30 produced when measuring the breath sample with desflurane against the ambient air reference are of the same magnitude as obtained in the case of desflurane in synthetic air, which indicates an almost complete removal of the volatile breath matrix components from the test gas.

The description of the preferred embodiments and the figures merely serve to illustrate the invention and the numerous advantages it entails, but should not be understood to indicate any limitation. The scope of the invention is determined solely by the appended claims.

### REFERENCE SIGNS

10 e-nose device
12 breathing circuit
14 patient under surgery
16 inlet line of breathing circuit 12
18 outlet line of breathing circuit 12
20 mouthpiece/nosepiece
22 respiratory valve
24 branch line
26 reference inlet
28 exhaust line
30 sensor array
32 nanoparticle network
34 nanoparticles
36 organic linker molecules
38 humidity sensors

1. A system (10) adapted for monitoring respiratory gases during anesthesia, comprising:
a sensor means comprising a sensor array (30) with a plurality of chemiresistive sensors; and
a conduct means adapted for supplying, to said chemiresistive sensors, an inhaled gas of a subject (14) and/or an exhaled gas of said subject (14);
wherein said sensor means are adapted to monitor said inhaled gas of said subject (14) and/or said exhaled gas of said subject (14) by analyzing a gas sample supplied via said conduct means to said chemiresistive sensors;
wherein said chemiresistive sensors comprise a nanoparticle film with a nanoparticle network (32), said nanoparticle network (32) formed of nanoparticles (34) interlinked through linker molecules (36); and
wherein a thickness of said nanoparticle film is no greater than 100 nm, preferably no greater than 50 nm.

2. The system (10) according to claim 1, wherein said nanoparticle network (32) in at least one of said chemiresistive sensors does not comprise a polymer.

3. The system (10) according to claim 1, wherein said linker molecules (36) in at least one of said chemiresistive sensors comprise non-linear polymer molecules or oligomer molecules, in particular dendrimer molecules.

4. The system (10) according to claim 1, wherein said nanoparticles (34) in said nanoparticle network (32) are arranged in a plurality of layers, wherein nanoparticles (34) of adjacent layers are connected by said linker molecules (36).

5. The system (10) according to claim 1, wherein said nanoparticles are metallic nanoparticles (34), and in particular comprise a metal selected from the group consisting of Au, Pt, Ag, Pd, Cu, Ni, Cr, Mo, Zr, Nb, Fe, or any combination of these metals.

6. The system (10) according to claim 1, wherein said sensor array (30) comprises a first group of chemiresistive sensors comprising hydrophilic linker molecules, and/or a second group of chemiresistive sensors comprising hydrophobic linker molecules, and/or a third group of chemiresistive sensors comprising amphiphilic linker molecules.

7. The system (10) according to claim 1, wherein said conduct means are adapted for sidestream connection to an inlet conduct (16), said inlet conduct (16) for supplying an anesthetic gas to said subject (14), and/or for sidestream connection to an outlet conduct (18), said outlet conduct (18) for removing exhaled breath from said subject (14).

8. The system (10) according to claim 1, wherein said conduct means further comprise a reference inlet (26) in fluid communication with said sensor array (30), said reference inlet (26) adapted for supplying a reference gas sample to said chemiresistive sensors.

9. The system (10) according to claim 1, wherein said sensor means comprise a humidity sensor (38) and/or a temperature sensor.

10. The system (10) according to claim 1, wherein said sensor means comprise analyzation means adapted to identify an anesthetic and/or analgesic in said inhaled gas and/or in said exhaled gas, and/or adapted to determine a concentration of an anesthetic and/or analgesic in said inhaled gas and/or said exhaled gas by determining a ratio of a signal provided by a first chemiresistive sensor to a signal provided by a second chemiresistive sensor, said second chemiresistive sensor different from said first chemiresistive sensor.

11. A method for monitoring respiratory gases during anesthesia, comprising the steps of:
providing a sensor means comprising a sensor array (30) with a plurality of chemiresistive sensors;
exposing said chemiresistive sensors to an inhaled gas of a subject (14) and/or to an exhaled gas of said subject (14); and
analyzing said inhaled gas and/or said exhaled gas;
wherein said chemiresistive sensors comprise a nanoparticle film with a nanoparticle network (32), said nanoparticle network (32) formed of nanoparticles (34) interlinked through linker molecules (36);
wherein a thickness of said nanoparticle film is no greater than 100 nm, preferably no greater than 50 nm.

12. The method according to claim 11, further comprising the steps of exposing said chemiresistive sensors to a reference gas sample, and comparing said inhaled gas and/or said exhaled gas to said reference gas sample.

13. The method according to claim 12, wherein said chemiresistive sensors are exposed to said reference gas sample after a predetermined number n of breathing cycles, each said breathing cycle comprising the steps of consecutively exposing said chemiresistive sensors to said inhaled gas of said subject (14) and to said exhaled gas of said subject (14).

14. The method according to claim 11, further comprising a step of identifying an anesthetic and/or analgesic in said inhaled gas and/or said exhaled gas, and/or a step of determining a concentration of an anesthetic and/or analgesic in said inhaled gas and/or said exhaled gas by determining a ratio of a signal provided by a first chemiresistive sensor to a signal provided by a second chemiresistive sensor, said second chemiresistive sensor being different from said first chemiresistive sensor.

15. A storage means adapted for storing computer-readable instructions, such that said instructions, when read on a computer connected to a system (10) for monitoring respiratory gases according to claim 1, implement on said system (10) a method according to claim 11.
